(19) United States
(12) Patent Application Publication
(10) Pub. No.: US 2005/0171365 A1
(43) Pub. Date: Aug. 4, 2005

(54) Epoxidation process using a mixed catalyst system

(76) Inventors: Roger A. Grey, (US); Edrick Morales, (US)

Correspondence Address: LYONDELL CHEMICAL COMPANY, 3801 WEST CHESTER PIKE, NEWTOWN SQUARE, PA, 19073, US

(21) Appl. No.: 10/770,924

(22) Filed: Feb. 3, 2004

(51) Int. Cl. C07D 301/06

(52) U.S. Cl. 549/533

(57) ABSTRACT

The invention is a process for epoxidizing olefins with hydrogen and oxygen in the presence of a palladium-containing titanium zeolite and a palladium-free titanium zeolite. The process exhibits good productivity and selectivity for olefin epoxidation with hydrogen, and oxygen. Surprisingly, the presence of palladium-free titanium zeolite in addition to the palladium-containing titanium zeolite in the process improves the palladium productivity of the process.

### FIELD OF THE INVENTION

This invention relates to an epoxidation process using a mixed catalyst system to produce epoxides from hydrogen, oxygen, and olefins. The mixed catalyst system comprises a palladium-containing titanium zeolite and a palladium-free titanium zeolite. Surprisingly, the presence of a palladium-free titanium zeolite in addition to the palladium-containing titanium zeolite results in enhanced productivity per amount of palladium.

### BACKGROUND OF THE INVENTION

Many different methods for the preparation of epoxides have been developed. Generally, epoxides are formed by the reaction of an olefin with an oxidizing agent in the presence of a catalyst. The production of propylene oxide from propylene and an organic hydroperoxide oxidizing agent, such as ethyl benzene hydroperoxide or tert-butyl hydroperoxide, is commercially practiced technology. This process is performed in the presence of a solubilized molybdenum catalyst, see U.S. Pat. No. 3,351,635, or a heterogeneous titania on silica catalyst, see U.S. Pat. No. 4,367,342. Hydrogen peroxide is another oxidizing agent useful for the preparation of epoxides. Olefin epoxidation using hydrogen peroxide and a titanium silicate zeolite is demonstrated in U.S. Pat. No. 4,833,260. One disadvantage of both of these processes is the need to pre-form the oxidizing agent prior to reaction with olefin.

Another commercially practiced technology is the direct epoxidation of ethylene to ethylene oxide by reaction with oxygen over a silver catalyst. Unfortunately, the silver catalyst has not proved useful in commercial epoxidation of higher olefins. Therefore, much current research has focused on the direct epoxidation of higher olefins with oxygen and hydrogen in the presence of a catalyst. In this process, it is believed that oxygen and hydrogen react in situ to form an oxidizing agent. Thus, development of an efficient process (and catalyst) promises less expensive technology compared to the commercial technologies that employ pre-formed oxidizing agents.

Many different catalysts have been proposed for use in the direct epoxidation of higher olefins. For example, JP 4-352771 and U.S. Pat. Nos. 5,859,265, 6,008,388, and 6,281,369 disclose the production of propylene oxide using titanium zeolite catalysts that incorporate a noble metal such as palladium. In addition, other catalysts disclosed include gold supported on titanium oxide, see for example U.S. Pat. No. 5,623,090, and gold supported on titanosilicates, see for example PCT Intl. Appl. WO 98/00413.

Mixed catalyst systems for olefin epoxidation with hydrogen and oxygen have also been disclosed. For instance, JP 4-352771 at Example 13 describes the use of a mixture of titanosilicate and Pd/C for propylene epoxidation. U.S. Pat. No. 6,498,259 describes a catalyst mixture of a titanium zeolite and a supported palladium complex, where palladium is supported on carbon, silica, silica-alumina, titania, zirconia, and niobia. Further, U.S. Pat. No. 6,441,204 describes a mixture of titanium zeolite and a palladium on niobium-containing support. In addition, U.S. Pat. No. 6,307,073 discloses a mixed catalyst system that is useful in olefin epoxidation comprising a titanium zeolite and a gold-containing supported catalyst, where gold is supported on supports such as zirconia, titania, and titania-silica.

One disadvantage of the described direct epoxidation catalysts is that they all show either less than optimal selectivity or productivity. As with any chemical process, it is desirable to attain still further improvements in the direct epoxidation methods and catalysts.

We have discovered an effective, convenient epoxidation catalyst mixture for use in the direct epoxidation of olefins with oxygen and hydrogen.

### SUMMARY OF THE INVENTION

The invention is an olefin epoxidation process that comprises reacting an olefin, oxygen, and hydrogen in the presence of a catalyst mixture comprising a palladium-containing titanium zeolite and a palladium-free titanium zeolite. The process surprisingly improves the palladium productivity of epoxidation compared to using just a palladium-containing titanium zeolite.

### DETAILED DESCRIPTION OF THE INVENTION

The process of the invention employs a catalyst mixture that comprises a palladium-containing titanium zeolite and a palladium-free titanium zeolite. Palladium-containing titanium zeolite catalysts are well known in the art and are described, for example, in JP 4-352771 and U.S. Pat. Nos. 5,859,265, 6,008,388, and 6,281,369, the teachings of which are incorporated herein by reference in their entirety. Such catalysts comprise palladium and a titanium zeolite. The palladium-containing titanium zeolite may also contain an additional noble metal, preferably platinum, gold, silver, iridium, rhenium, ruthenium, or osmium; and most preferably, platinum or gold.

Both the palladium-containing titanium zeolite and the palladium-free titanium zeolite contain a titanium zeolite. Titanium zeolites comprise the class of zeolitic substances wherein titanium atoms are substituted for a portion of the silicon atoms in the lattice framework of a molecular sieve. Such substances are well known in the art. Particularly preferred titanium zeolites include the class of catalysts commonly referred to as titanium silicalites, particularly “TS-1” (having an MFI topology analogous to that of the ZSM-5 aluminosilicate zeolites), “TS-2” (having an MEL topology analogous to that of the ZSM-11 aluminosilicate zeolites), and “TS-3” (as described in Belgian Pat. No. 1,001,038). Titanium-containing catalysts having framework structures isomorphous to zeolite beta, mordenite, ZSM-48, ZSM-12, and MCM-41 are also suitable for use. The titanium zeolites preferably contain no elements other than titanium, silicon, and oxygen in the lattice framework, although minor amounts of boron, iron, aluminum, sodium, potassium, copper and the like may be present.

The typical amount of palladium present in the palladium-containing titanium zeolite will be in the range of from about 0.01 to 20 weight percent, preferably 0.01 to 10 weight percent, and particularly 0.03 to 5 weight percent. The manner in which the palladium is incorporated into the catalyst is not considered to be particularly critical. For example, the palladium may be supported on the zeolite by impregnation or the like. Alternatively, the palladium can be incorporated into the zeolite by ion-exchange with, for example, Pd tetraamine chloride.

There are no particular restrictions regarding the choice of palladium compound used as the source of palladium. For example, suitable compounds include the nitrates, sulfates, halides (e.g., chlorides, bromides), carboxylates (e.g. acetate), and amine complexes of palladium. The palladium may be in an oxidation state anywhere from 0 to +4 or any combination of such oxidation states. To achieve the desired oxidation state or combination of oxidation states, the palladium compound may be fully or partially pre-reduced after addition to the catalyst. Satisfactory catalytic performance can, however, be attained without any pre-reduction. To achieve the active state of palladium, the palladium-containing titanium zeolite may undergo pretreatment such as thermal treatment in nitrogen, vacuum, hydrogen, or air.

If the palladium-containing titanium zeolite contains an additional noble metal such as platinum, gold, silver, iridium, rhenium, ruthenium, or osmium, the amount of noble metal will typically be in the range of from about 0.001 to 10 weight percent, and preferably 0.01 to 5 weight percent. The manner in which the additional noble metal is incorporated into the catalyst is not considered to be particularly critical. The additional noble metal may be added to the titanium zeolite using the same techniques used to incorporate palladium. The additional noble metal may be added before, during, or after palladium incorporation.

The process of the invention also employs a palladium-free titanium zeolite. By “palladium-free”, we mean that the titanium zeolite is free of added palladium. The palladium-free titanium zeolite may be the same zeolite that makes up part of the palladium-containing titanium zeolite of the invention, or they may be different.

The palladium-containing titanium zeolite and a palladium-free titanium zeolite may be used in the epoxidation process as a mixture of powders or as a mixture of pellets. In addition, the palladium-containing titanium zeolite and the palladium-free titanium zeolite may also be pelletized or extruded together prior to use in epoxidation. If pelletized or extruded together, the catalyst mixture may additionally comprise a binder or the like and may be molded, spray dried, shaped or extruded into any desired form prior to use in epoxidation. The weight ratio of palladium-containing titanium zeolite:palladium-free titanium zeolite is not particularly critical. However, a palladium-containing titanium zeolite:palladium-free titanium zeolite ratio of 0.01-100 (grams of palladium-containing titanium zeolite per gram of palladium-free titanium zeolite) is preferred, and 0.1-10 is particularly preferred.

The mixture of a palladium-containing titanium zeolite and a palladium-free titanium zeolite is useful for catalyzing the epoxidation of olefins with oxygen and hydrogen. This epoxidation process comprises contacting an olefin, oxygen, and hydrogen in the presence of the catalyst mixture. Suitable olefins include any olefin having at least one carbon-carbon double bond, and generally from 2 to 60 carbon atoms. Preferably the olefin is an acyclic alkene of from 2 to 30 carbon atoms; the process of the invention is particularly suitable for epoxidizing C₂-C₆ olefins. More than one double bond may be present, as in a diene or triene for example. The olefin may be a hydrocarbon (i.e., contain only carbon and hydrogen atoms) or may contain functional groups such as halide, carboxyl, hydroxyl, ether, carbonyl, cyano, or nitro groups, or the like. The process of the invention is especially useful for converting propylene to propylene oxide.

Oxygen and hydrogen are also required for the epoxidation process. Although any sources of oxygen and hydrogen are suitable, molecular oxygen and molecular hydrogen are preferred.

Epoxidation according to the invention is carried out at a temperature effective to achieve the desired olefin epoxidation, preferably at temperatures in the range of 0-250° C., more preferably, 20-100° C. The molar ratio of hydrogen to oxygen can usually be varied in the range of H₂:O₂=1:10 to 5:1 and is especially favorable at 1:5 to 2:1. The molar ratio of oxygen to olefin is usually 2:1 to 1:20, and preferably 1:1 to 1:10. Relatively high oxygen to olefin molar ratios (e.g., 1:1 to 1:3) may be advantageous for certain olefins. A carrier gas may also be used in the epoxidation process. As the carrier gas, any desired inert gas can be used. The molar ratio of olefin to carrier gas is then usually in the range of 100:1 to 1:10 and especially 20:1 to 1:10.

As the inert gas carrier, noble gases such as helium, neon, and argon are suitable in addition to nitrogen and carbon dioxide. Saturated hydrocarbons with 1-8, especially 1-6, and preferably with 1-4 carbon atoms, e.g., methane, ethane, propane, and n-butane, are also suitable. Nitrogen and saturated C₁-C₄ hydrocarbons are the preferred inert carrier gases. Mixtures of the listed inert carrier gases can also be used.

Specifically in the epoxidation of propylene, propane can be supplied in such a way that, in the presence of an appropriate excess of carrier gas, the explosive limits of mixtures of propylene, propane, hydrogen, and oxygen are safely avoided and thus no explosive mixture can form in the reactor or in the feed and discharge lines.

The amount of palladium-containing titanium zeolite and palladium-free titanium zeolite used may be varied according to many factors, including the amount of palladium contained in the palladium-containing titanium zeolite. The total amount of catalyst mixture may be determined on the basis of the molar ratio of the titanium (contained in the palladium-containing titanium zeolite and the palladium-free titanium zeolite) to the olefin that is supplied per unit time. Typically, sufficient catalyst mixture is present to provide a titanium/olefin feed ratio of from 0.0001 to 0.1 hour. The time required for the epoxidation may be determined on the basis of the gas hourly space velocity, i.e., the total volume of olefin, hydrogen, oxygen and carrier gas(es) per unit hour per unit of catalyst volume (abbreviated GHSV). A GHSV in the range of 10 to 10,000 hr⁻¹ is typically satisfactory.

Depending on the olefin to be reacted, the epoxidation according to the invention can be carried out in the liquid phase, the gas phase, or in the supercritical phase. When a liquid reaction medium is used, the catalyst is preferably in the form of a suspension or fixed-bed. The process may be performed using a continuous flow, semi-batch or batch mode of operation.

If epoxidation is carried out in the liquid (or supercritical) phase, it is advantageous to work at a pressure of 1-100 bars and in the presence of one or more solvents. Suitable solvents include, but are not limited to, alcohols, water, supercritical CO₂, or mixtures thereof. Suitable alcohols include C₁-C₄ alcohols such as methanol, ethanol, isopropanol, and tert-butanol, or mixtures thereof. Fluorinated alcohols can be used. It is preferable to use mixtures of the cited alcohols with water.

If epoxidation is carried out in the liquid (or supercritical) phase, it is advantageous to use a buffer. The buffer will typically be added to the solvent to form a buffer solution. The buffer solution is employed in the reaction to inhibit the formation of glycols during epoxidation. Buffers are well known in the art.

Buffers useful in this invention include any suitable salts of oxyacids, the nature and proportions of which in the mixture, are such that the pH of their solutions may range from 3 to 10, preferably from 4 to 9 and more preferably from 5 to 8. Suitable salts of oxyacids contain an anion and cation. The anion portion of the salt may include anions such as phosphate, carbonate, bicarbonate, carboxylates (e.g., acetate, phthalate, and the like), citrate, borate, hydroxide, silicate, aluminosilicate, or the like. The cation portion of the salt may include cations such as ammonium, alkylammoniums (e.g., tetraalkylammoniums, pyridiniums, and the like), alkali metals, alkaline earth metals, or the like. Cation examples include NH₄, NBu₄, NMe₄, Li, Na, K, Cs, Mg, and Ca cations. More preferred buffers include alkali metal phosphate and ammonium phosphate buffers. Buffers may preferably contain a combination of more than one suitable salt. Typically, the concentration of buffer in the solvent is from about 0.0001 M to about 1 M, preferably from about 0.001 M to about 0.3 M. The buffer useful in this invention may also include the addition of ammonia gas to the reaction system.

An epoxide product is produced by the process of the invention.

The following examples merely illustrate the invention. Those skilled in the art will recognize many variations that are within the spirit of the invention and scope of the claims.

### EXAMPLE 1

### Catalyst Preparation

Catalyst 1A: A spray dried TS-1 (112 g, 80% TS-1, 20% silica; 1.7 wt. % Ti) is calcined in air at 550° C., placed in a round bottom flask, and then slurried in deionized water (250 mL). To the slurry, an aqueous solution of Pd(NH₃)₄Cl₂ (1.3 g in 90 g of deionized water) is added with mixing over 30 minutes. The slurry is mixed on a rotoevaporator at 30 rpm in a 30° C. water bath for an additional 2 hours. The solids are isolated by filtration and the filter cake is washed by re-slurrying in deionized water (140 mL) and filtering again. The washing is conducted four times. The solids are air dried overnight and dried in a vacuum oven at 50° C. for 8 hours. By elemental analysis, the dried material contains 0.34 wt. % Pd and 1.67 wt. % titanium; residual chloride was less than 20 ppm.

The dried solids are air calcined in an oven by heating to 110° C. (at 10° C./min) and holding at 110° C. for 4 hours, then heating to 150° C. (at 2° C./min) and holding at 150° C. for 4 hrs. The calcined solids are transferred to a quartz tube and treated with hydrogen (5% in nitrogen; 100 mL/min) at 50° C. for 4 hours followed by nitrogen only for one hour before cooling to room temperature and isolating Catalyst 1A.

Catalyst 1B: Catalyst 1B was prepared according to the same procedure as for Catalyst 1A, except that the aqueous solution of Pd(NH₃)₄Cl₂ contains only 0.45 g Pd(NH₃)₄Cl₂ in 30 g of deionized water. Catalyst 1B contains 0.11 wt. % Pd and 1.7 wt. % titanium; residual chloride was less than 20 ppm.

Catalyst 1C: TS-1 powder (2.2 wt % Ti, calcined at 550° C. in air) is slurried in deionized water (100 grams). A solution of palladium acetate (0.5 g in 50 mL of acetone) is added under nitrogen to the slurry over a 5 minute period, then the mixture is turned on a rotoevaporator (30 rpm) under nitrogen for 30 minutes at 23° C. and 4 hours at 50° C. About one half of the liquid is removed under vacuum, then the solids are isolated by filtration, washed two times with 50 grams of deionized water and dried at 110° C. for 4 hours. By elemental analysis, the dried material contains 0.4 wt. % Pd and 2.17 wt. % Ti.

The solids are transferred to a quartz tube and treated with hydrogen (5% in nitrogen; 100 mL/min) at 60° C. for 2 hours followed by nitrogen only for one hour before cooling to room temperature and isolating Catalyst 1C.

### EXAMPLE 2

### Buffer Preparation

Buffer 2A—0.1 Molar pH 6 Ammonium Phosphate Buffer: Ammonium dihydrogen phosphate (NH₄H₂PO₄, 11.5 g) is dissolved in deionized water (900 g). Aqueous ammonium hydroxide (30% NH₄OH) is then added to the solution until the pH reads 6 via a pH meter. The volume of the solution is then increased to 1000 mL by addition of deionized water.

Buffer 2B—0.2 Molar pH 7 Ammonium Phosphate Buffer: Ammonium dihydrogen phosphate (23 g) is dissolved in deionized water (900 g). Aqueous ammonium hydroxide (30% NH₄OH) is then added to the solution until the pH reads 7 via a pH meter. The volume of the solution is then increased to 1000 mL by addition of deionized water.

### EXAMPLE 3

### Propylene Epoxidation in MEOH/Water

### Example 3A

A 300 cc stainless steel reactor is charged with Catalyst 1A (0.2 g), spray dried TS-1 (0.5 g, 80% TS-1, 20% silica; 1.7 wt. % Ti), Buffer 2A (13 g), and methanol (100 g). The reactor is then charged to 300 psig with a feed consisting of 2 vol. % H₂, 4 vol. % O₂, 5 vol. % propylene, 0.5 vol. % methane and the balance nitrogen. The reactor pressure is maintained at 300 psig via a back pressure regulator with the feed gases passed continuously through the reactor at 1600 cc/min (measured at 21° C. and one atmosphere pressure). In order to maintain a constant solvent level in the reactor during the run, the oxygen, nitrogen and propylene feeds are first passed through a two-liter stainless steel vessel (saturator) containing 1.5 liters of methanol prior to the reactor. The reactor is stirred at 1500 rpm and the reaction mixture is heated to 60° C. The gaseous effluent is analyzed by an online GC every hour and the liquid analyzed by offline GC at the end of the 18 hour run. The results are shown in Table 1.

### Comparative Example 3B

Comparative example 3B is conducted according to the procedure of Example 3A, except that 0.7 gram of Catalyst 1 B is used as the only catalyst. The results are shown in Table 1.

### EXAMPLE 4

### Propylene Epoxidation in Water

### Example 4A

A one-liter stainless steel reactor is charged with Catalyst 1C (12 g), TS-1 powder (12 g, 2.2 wt % Ti, calcined at 550° C. in air), and Buffer 2B (376 g). The reactor is then charged to 500 psig with a feed consisting of 4 vol. % H₂, 4 vol. % O₂, 27 vol. % propylene, 0.5 vol. % methane and the balance nitrogen. The reactor pressure is maintained at 500 psig via a back pressure regulator with the feed gases passed continuously through the reactor at 405 L/h (measured at 21° C. and one atmosphere pressure). The reactor is stirred at 500 rpm, and the reaction mixture is heated to 60° C. The gaseous effluent is analyzed by an online GC every hour and the liquid analyzed by offline GC at the end of the 18 hour run. The results are shown in Table 1.

### Comparative Example 4B

Comparative example 4B is conducted according to the procedure of Example 4A, except that the reactor is charged with Catalyst 1C (12 g) and Buffer 2B (388 g) only. The results are shown in Table 1.

The results show that there is an unexpected advantage to using a catalyst mixture (Pd/TS-1 plus TS-1) compared to using Pd/TS-1 only. The palladium in the catalyst mixture produces a greater amount of epoxide compared to the palladium in a Pd/TS-1 catalyst alone. The methanol run of Example 3, for instance, shows 17% higher palladium productivity and the water run of Example 4 shows 36% higher palladium productivity. In addition to the higher palladium productivity, there may be an economic advantage in catalyst synthesis. As demonstrated in Example 3, only a fraction of the overall TS-1 needs to undergo palladium incorporation (although a higher amount of palladium is needed) and the addition of palladium-free TS-1 can still result in slightly higher productivity. This observation can result in economic savings by requiring the processing of less TS-1 in palladium incorporation. Also, the PO/POE selectivity is unaffected, or slightly improved, when using the catalyst mixture. “POE” means PO equivalents which include propylene oxide (PO), propylene glycol (PG), dipropylene glycol (DPG), 1-methoxy-2-propanol (PM-1), 2-methoxy-1-propanol (PM-2), and acetol.

TABLE 1
COMPARISON OF CATALYST ACTIVITY

| | | Wt. of | Wt. of | | Total | |
|---|---|---|---|---|---|---|
| Ex- | | Total | Pd in | PO/POE | Catalyst | Palladium |
| ample | | Catalyst | catalyst | Selectivity | Pro- | Pro- |
| # | Catalyst | (g) | (mg) | (%)¹ | ductivity² | ductivity³ |
| 3A | 1A + | 0.7 | 0.68 | 93 | 0.33 | 340 |
| | TS-1 | | | | | |
| 3B* | 1B | 0.7 | 0.77 | 93 | 0.32 | 290 |
| 4A | 1C + | 24 | 48 | 81 | 0.051 | 26 |
| | TS-1 | | | | | |
| 4B* | 1C | 12 | 48 | 76 | 0.076 | 19 |

*Comparative Example
¹PO/POE Selectivity = moles PO/(moles P0 + moles propylene glycols) * 100.
²Total Catalyst Productivity = grams POE produced/gram of total catalyst per hour.
³Palladium Productivity = grams POE produced/gram of palladium per hour.

1. A process for producing an epoxide comprising reacting an olefin, hydrogen and oxygen in the presence of a catalyst mixture comprising a palladium-containing titanium zeolite and a palladium-free titanium zeolite.

2. The process of claim 1 wherein the palladium-containing titanium zeolite comprises palladium and a titanium silicalite.

3. The process of claim 2 wherein the titanium silicalite is TS-1.

4. The process of claim 1 wherein the palladium-containing titanium zeolite comprises palladium, a titanium zeolite, and a noble metal selected from the group consisting of platinum, gold, silver, iridium, rhenium, ruthenium, osmium, and mixtures thereof.

5. The process of claim 4 wherein the noble metal is selected from the group consisting of platinum, gold, and mixtures thereof.

6. The process of claim 1 wherein the palladium-containing titanium zeolite comprises from about 0.01 to about 10 weight percent palladium.

7. The process of claim 1 wherein the palladium-free titanium zeolite is a titanium silicalite.

8. The process of claim 1 wherein the palladium-free titanium zeolite is TS-1.

9. The process of claim 1 wherein the olefin is a C₂-C₆ olefin.

10. The process of claim 1 wherein the olefin is propylene.

11. The process of claim 1 wherein reaction of olefin, hydrogen and oxygen is performed in a solvent.

12. The process of claim 11 wherein the solvent is selected from the group consisting of water, C₁-C₄ alcohols, supercritical CO₂, and mixtures thereof.

13. The process of claim 11 wherein the solvent contains a buffer.

14. A process comprising reacting propylene, hydrogen and oxygen in a solvent in the presence of a catalyst mixture comprising a palladium-containing titanium silicalite and palladium-free TS-1, wherein the palladium-containing titanium silicalite comprises palladium and a titanium silicalite.

15. The process of claim 14 wherein the titanium silicalite is TS-1.

16. The process of claim 14 wherein the palladium-containing titanium zeolite further comprises a noble metal selected from the group consisting of platinum, gold, silver, iridium, rhenium, ruthenium, osmium, and mixtures thereof.

17. The process of claim 14 wherein the solvent is selected from the group consisting of water, C₁-C₄ alcohols, supercritical CO₂, and mixtures thereof.

18. The process of claim 14 wherein the solvent contains a buffer.

19. (canceled)

20. (canceled)
